(19) United States
(12) Patent Application Publication
(10) Pub. No.: US 2009/0111856 A1
(43) Pub. Date: Apr. 30, 2009

(54) Parenteral Formulation Comprising Proton Pump Inhibitor Sterilized in its Final Container by Ionizing Radiation

(76) Inventors: Mikael Brulls, Molndal (SE); Johanna Karlsson, Molndal (SE)

Correspondence Address: WHITE & CASE LLP;PATENT DEPARTMENT, 1155 AVENUE OF THE AMERICAS, NEW YORK, NY, 10036, US

(21) Appl. No.: 12/299,684

(22) PCT Filed: May 7, 2007
(86) PCT No.: PCT/SE2007/000440

§ 371 Date: Nov. 5, 2008

Related U.S. Application Data
(60) Provisional application No. 60/799,058, filed on May 9, 2006.

(51) Int. Cl. A61K 31/435; A61P 1/00

(52) U.S. Cl. 514/338

(57) ABSTRACT

The present invention relates to a stable sterilized parenteral formulation comprising an acid susceptible proton pump inhibitor. The formulation, a solid formulation comprising the acid susceptible proton pump inhibitor and optionally pharmaceutically acceptable excipients, has been sterilized in its final container by ionizing radiation. The container may consist of several compartments and separately contains a suitable solvent, which is sterilized, i.e. radiated, at the same time as the solid formulation. Alternatively, the suitable solvent is sterilized separately or aseptically manufactured. The solid formulation is dissolved in a suitable solvent before being administered to the patient, i.e. being prepared ex tempore. The present invention also relates to the prepared sterilized parenteral formulation, the stable solid formulation, processes for obtaining said parenteral formulation and the solid formulation as well as to the therapeutic uses thereof.

### FIELD OF THE INVENTION

The present invention relates to a stable sterilized parenteral formulation comprising an acid susceptible proton pump inhibitor. The formulation, a solid formulation for parenteral use comprising the acid susceptible proton pump inhibitor and optionally pharmaceutically acceptable excipients, is sterilized in its final container by ionizing radiation. The container may consist of several compartments, one of which contains separately a suitable solvent, which is sterilized, i.e. radiated, at the same time as the solid formulation contained separately in the other compartment of the container. Alternatively, the suitable solvent is sterilized separately or manufactured aseptically. The solid formulation for parenteral use is dissolved in a suitable solvent before being administered to the patient, i.e. being prepared ex tempore. The present invention also relates to the prepared stable sterilized parenteral formulation as such, the stable solid formulation as such, processes for obtaining said parenteral formulation as well as to the therapeutic uses thereof.

### BACKGROUND OF THE INVENTION AND PRIOR ART

It is known in the art that gamma radiation can be used for sterilization. See for instance, WO 04/037224, which describes an injectable depot formulation in the form of a suspension comprising an aryl-heterocyclic compound, a viscosity agent and a solubilizer, such as cyclodextrin. Gamma radiation is mentioned as a sterilization method for the formulation.

Spray-drying of a proton pump inhibitor compound from absolute ethanol solution has been used to prepare amorphous forms of pantoprazole sodium hydrates (International Journal of Pharmaceutics 292 (2005) 59-68), and sodium pantoprazole-loaded enteric coated microparticles have been prepared by spray-drying using a polymer solution (International Journal of Pharmaceutics 324 (2006) 10-18). Spray drying from ethanol solutions has been used as one possible method to obtain inclusion complex between omeprazole and γ-cyclodextrin (Arias et al, Drug Development and Industrial Pharmacy 26(3), p 253-259 (2000)).

U.S. Pat. No. 6,331,174 B1 relates to a pre-filled disposable syringe for injection, which syringe avoids glass as a construction material. The syringe is designed to be resistant to gamma rays.

EP 1369130 A1 relates to a process for producing sustained release preparations of a poorly water-soluble non-peptidic physiologically active compound in an organic solvent solution of a biodegradable polymer in an amount higher than the solubility of the compound. In order to prepare a sterile preparation of the obtained sustained release preparation a method for sterilization with γ-ray may be employed. It is also mentioned in the patent specification that the prepared sustained release preparation of a poorly water-soluble non-peptidic compound may be co-administered together with other drugs. The list of possible drugs for co-administration mentions proton pump inhibitors, such as lansoprazole. However, there is no disclosure or proposal that the drugs, which may be co-administered with the produced sustained release preparation of a poorly water-soluble non-peptidic compound would be subject to any sterilization step.

WO97/09026 relates to a method for aseptic and automatic transfer of unsealed pharmaceutical containers, which have been aseptically filled with a pharmaceutical preparation.

Proton pump inhibitors are sensitive to heat and light and susceptible to chemical degradation in liquid solutions. The chemical degradation is pH-dependent and the rate of reaction is very high at low pH values. Formulations for parenteral administration comprising proton pump inhibitor compounds are due to their chemical susceptibility formulated as solid formulations for ex tempore reconstitution in a sterile solvent just before use. These solid formulations have so far been obtained by lyophilisation of a sterile filtered and aseptically filled solution. Lyophilisation is a process where the material (in this case the solution) is freeze-dried in a vacuum to vaporize the frozen water. The resulting product is a porous cake or powder. Lyophilisation is a complex and time consuming process, and hence very expensive.

The chemical instability of the proton pump inhibitors precludes heat sterilization of this class of compounds. These compounds must also be protected from light because of their light sensitivity.

Proton pump inhibitors are for instance compounds known under the nonproprietary names omeprazole, lansoprazole, pantoprazole, rabeprazole, leminoprazole and esomeprazole. Omeprazole and therapeutically acceptable salts thereof are described in EP-A1-0005129. EP-A1-124495 describes certain salts of omeprazole and EP-A1-174726, EP-A1-166287 and GB 2163747 are directed to lansoprazole, pantoprazole and rabeprazole, respectively. WO 94/27988 is directed to salts of the single enantiomers of omeprazole, including pharmaceutically acceptable alkaline salts of esomeprazole such as sodium and magnesium salts.

WO 94/02141 describes an injection of an antiulcerative benzimidazole compound, such as omeprazole. The injection comprises a lyophilized product, which is dissolved in physiological saline just before use. The lyophilized product is prepared from the sodium salt of omeprazole together with sodium hydroxide using water as the solvent.

WO 05/058277 describes an injectable formulation comprising lansoprazole and a chelating agent, and WO 05/065682 describes a parenteral formulation of rabeprazole.

WO 01/28558 describes an alternative type of parenteral formulations, which is not freeze-dried. These formulations are water free or almost water free, stable liquid formulations comprising polyethylene glycol and a sodium or potassium salt of the active ingredient.

Formulations intended for parenteral administration should comprise an active compound with satisfactory aqueous solubility. The formulations must also have and maintain suitable storage stability, and should be easy to handle and inexpensive to manufacture.

The present invention provides stable solid formulations suitable for parenteral administration after ex tempore reconstitution in a sterile solvent, without using any lyophilisation processes/steps in the manufacturing process of the formulation.

It has surprisingly been found that it is possible to sterilize by ionizing radiation a solid formulation comprising an acid susceptible proton pump inhibitor compound, which is sensitive to light exposure.

### OUTLINE OF THE PRESENT INVENTION

The present invention relates to a stable sterilized parenteral formulation comprising an acid susceptible proton pump inhibitor and optionally pharmaceutically acceptable excipients wherein said formulation is sterilized in its final container by ionizing radiation. The sterilized stable solid composition in said container or in another suitable package can be stored at room temperature and/or at elevated temperatures. Such a sterilized stable solid formulation is suitable for an ex tempore preparation of a solution for parenteral administration.

According to one embodiment of the present invention, the product is a multi-compartment container comprising in separate compartments a stable solid formulation and a suitable solvent, respectively. This product is sterilized by radiation. Before administration of the parenteral formulation, the wall between the separate compartments will be broken and an ex tempore prepared solution for parenteral administration is formed.

Alternatively, the product is a single compartment container comprising a stable solid formulation. This product is sterilized by radiation. Before administration a suitable solvent can be added to this product, i.e. to the single compartment container, to form an ex tempore solution for parenteral administration.

The present invention also relates to a stable solid formulation comprising an acid susceptible proton pump inhibitor and optionally pharmaceutically acceptable excipients wherein said solid formulation has been sterilized by ionizing radiation.

The invention also relates to an ex tempore prepared solution of the sterilized stable solid formulation comprising an acid susceptible proton pump inhibitor and optionally pharmaceutically acceptable excipients. Such a solution for parenteral administration is prepared by mixing the sterilized stable solid formulation with a suitable sterile solvent.

A suitable solvent for preparation of the ex tempore solution suitable for parenteral administration is for instance an aqueous solvent, such as physiological saline. The solvent must be sterile and aseptically filled into the final container before administration.

Alternatively, the solvent and the stable solid formulation, present in separate compartments, are sterilized in the final container.

The ex tempore prepared solution for parenteral administration must be free or essentially free from particles. The final container for administration of the parenteral formulation may therefore also have a particle filter incorporated in its construction. As discussed below, a solution filtration step to remove possible particle contamination followed by a spray drying step may be used in the preparation of the stable solid formulation according to one aspect of the invention.

The term “sterilized stable formulation” is intended to include formulations that show no or almost no significant degradation during storage (i.e. the degradation is approximately at the same level as for not sterilized starting material).

The term “ionizing radiation” is intended to include, unless stated otherwise, gamma radiation, electronic beam radiation and X-ray radiation. According to one embodiment of the invention, gamma radiation is used for the sterilization. According to another embodiment, electronic beam is used for the sterilization. According to a further embodiment, X-ray is used for the sterilization. For sterilization by gamma or electronic beam radiation doses up to about 45 kGy, e.g. 10 to 40 kGy, are used and preferably about 25 kGy. If the stable solid formulation and optional solvent are in its final container, it is important that the radiation penetrates the container and its complete content, i.e. the solid formulation and an optional solvent.

Thus, the material of the container may be critical for the result of the present invention and it should be radiation resistant.

Pharmaceutically acceptable excipients used in the present invention are selected from lactose, dextran, sodium chloride, polyvidone, cyclodextrines or amino acids such as arginine, cysteine, glycine, histidine, methionine or lysine or the like. It may be critical to select excipients, which do not show any or only small discoloration after radiation and insignificant degradation. Thus, also other pharmaceutically inactive excipients can be used, as long as the said excipient does not significantly change properties during or after radiation, neither chemically nor physically.

One embodiment of the present invention discloses that the acid susceptible proton pump inhibitor is selected from a compound of formula (I)

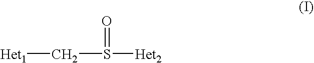

wherein

Het₁ is

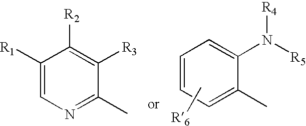

Het₂ is

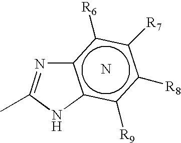

wherein

N in the benzimidazole moiety means that one of the carbon atoms substituted by R₆-R₉ optionally may be exchanged for a nitrogen atom without any substituents;

R₁, R₂ and R₃ are the same or different and selected from hydrogen, alkyl, alkoxy optionally substituted by fluorine, alkylthio, alkoxyalkoxy, dialkylamino, piperidino, morpholino, halogen, phenyl and phenylalkoxy;

R₄ and R₅ are the same or different and selected from hydrogen, alkyl and aralkyl;

R′₆ is hydrogen, halogen, trifluoromethyl, alkyl and alkoxy;

R₆-R₉ are the same or different and selected from hydrogen, alkyl, alkoxy, halogen, halo-alkoxy, alkylcarbonyl, alkoxycarbonyl, oxazolyl, pyrrolyl, trifluoroalkyl, or adjacent groups R₆-R₉ form ring structures;

or an enantiomer thereof.

Alkyl groups, alkoxy groups and moieties thereof in the definitions above may be branched or straight C₁-C₉-chains or comprise cyclic alkyl groups, such as cycloalkylalkyl;

Examples of proton pump inhibitors according to formula (I) are

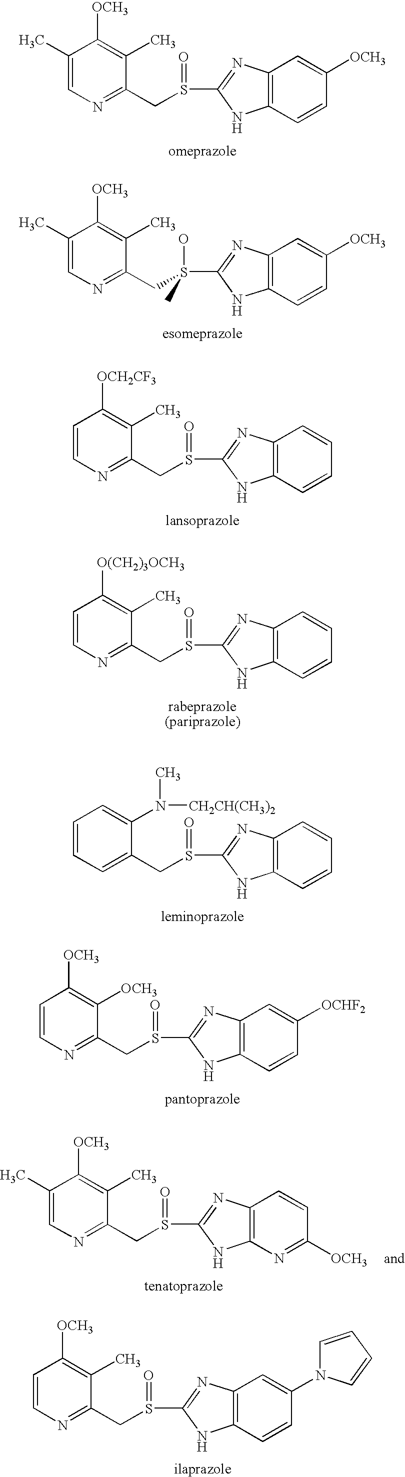

The acid susceptible proton pump inhibitors used in the sterilized parenteral formulation of the present invention may be used in their neutral form or in the form of a pharmaceutically acceptable salt such as an alkaline salt, which is soluble in water selected from any one of their, Na⁺, K⁺, Li⁺ or TBA (tert-butyl ammonium) salts.

Further, any given chemical formula or name shall encompass all stereo and optical isomers and racemates thereof as well as mixtures in different proportions of the separate enantiomers, where such isomers and enantiomers exist, as well as pharmaceutically acceptable salts thereof and solvates thereof, such as for instance hydrates. The above-listed compounds can also be used in their tautomeric form. Also included in the present invention are derivatives of the compounds listed above, which have the biological function of the compounds listed, such as prodrugs, see for instance US 2005/0182101.

The above exemplified proton pump inhibitors are for example disclosed in EP-A1-0005129, EP-A1-174 726, EP-A1-166 287, GB 2 163 747 and WO90/06925, WO91/19711, WO91/19712, WO98/54171, WO94/27988, WO98/54171 and WO00/044744. Suitable processes for the preparation of single enantiomers of the above proton pump inhibitor compounds are described in for instance WO96/02535, WO97/02261 and WO04/035565.

The acid susceptible proton pump inhibitor should have a satisfactory solubility in aqueous solvents, i.e. being soluble or sparingly soluble according to Ph Eur 2005. The proton pump inhibitor compound is either used in the present invention in its neutral, i.e. non-salt, form or in a pharmaceutically acceptable salt form including solvates such as hydrates.

The terms “soluble” and “sparingly soluble” are defined in accordance with the European Pharmacopoeia (Ph Eur 2005).

According to one embodiment of the present invention the compound of formula (I) or a separate single enantiomer thereof is incorporated in the form of a pharmaceutically acceptable salt in the claimed sterilized parenteral formulation and sterilized solid formulation.

In another embodiment of the present invention said pharmaceutically acceptable salt is sodium salt or potassium salt of esomeprazole including solvates, such as hydrates thereof. In another embodiment the pharmaceutically acceptable salt is sodium salt or potassium salt of omeprazole including solvates, such as hydrates thereof.

The present invention also relates to a process for manufacturing a parenteral formulation in its final container comprising the following steps: (i) filling a container with an acid susceptible proton pump inhibitor (in solid state) and optionally pharmaceutically acceptable excipients under controlled environment conditions, and (ii) sterilizing the pre-filled container by using ionizing radiation. Said container comprises for instance sodium or potassium salt of a compound of formula (I), which has a suitable water solubility.

In this embodiment, the container must be radiation resistant, i.e. not significantly change properties during or after radiation, neither chemically nor physically. One example of a suitable container for the present invention is, but not limited to, a vial made of radiation resistant material, such as radiation resistant glass. Radiation resistant glass typically contains cerium oxide, which prevents the glass from changing properties after radiation. In contrast, normal borosilicate glass typically turns brown after radiation. Alternatively, the container may be prepared from radiation resistant polypropylene, polyethylene or any other suitable material or combinations thereof.

One example could be a two-chamber bag where the two compartments are separated by a weak seal and comprises the drug and solvent in separate, pre-filled compartments for ex tempore preparation of a solution for parenteral administration. The weak seal breaks by applying pressure, e.g. via hands, on the compartment containing the solvent, allowing complete mixing of the drug and the solvent within the closed system. Thus, the product is sterilized with ionizing radiation in its final container.

The material used in the container shall be radiation resistant, i.e. not significantly changes properties during or after radiation, neither chemically nor physically. Examples of critical parameters for the function of the two-chamber bag are e.g. water barrier properties, seal strength, flexibility, tensile strength, transparency and visual appearance. Special considerations should be taken to the properties of the weak seal, e.g. seal strength, barrier properties and opening. It is important that the properties of the weak seal are not significantly affected by the radiation.

It has been demonstrated that ionizing radiation has no significant influence on the seal strength of the weak seal on bags made of a polypropylene based film.

The container material can additionally (especially over the powder compartment) be covered by a high barrier material, such as aluminum foil, to avoid light exposure to the active ingredient and/or exposure to e.g. moisture, oxygen and/or carbon dioxide. It has also been demonstrated that it is possible to weld an aluminum foil/laminate onto the polypropylene based film in a peel able as well as permanent way without significantly influence on the properties of the weak seal.

The container can further be placed in another pack that is made of e.g. aluminum or any other suitable material. The container may be sterilized after it has been placed in its final pack.

Filling of the container with the proton pump inhibitor compound should be done under controlled conditions, such as under controlled room temperate and dry conditions, due to the sensitivity of the proton pump inhibitor compound.

The present invention also relates to a process for the preparation of any of the parenteral formulations and solid formulations wherein the acid susceptible proton pump inhibitor is optionally mixed with pharmaceutically acceptable excipient(s) where after the formulation as such or in its final container is radiated with ionizing radiation. The formulations can be either non-lyophilized or lyophilized. Under certain circumstance a lyophilized formulation can be used. For instance a final container, which is pre-filled with a lyophilized solid formulation and a suitable solvent, is sterilized.

To facilitate the manufacturing it is advantageous to use a non-lyophilized solid formulation to obtain superior storage stability and to have enhanced properties such as better flow ability of the solid formulation when it is filled in its final container before the sterilization by ionizing radiation. According to one embodiment of the present invention the solid formulation is non-lyophilized and it is filled in its final container before it is sterilized by radiation. The sterilized formulation is suitable for an ex tempore preparation of a solution for parenteral administration.

The solid formulation may optionally be prepared by first dissolving a dry powder of an acid susceptible proton pump inhibitor compound and an optional pharmaceutically acceptable excipient in water or an ethanol solution and then drying the formulation in a suitable spray-dryer (See example 4). Alternatively, the different components may be dissolved in water or an ethanol solution separately and then spray-dried. Finally, the components of the solid formulation are mixed together.

In Example 4 below, spray drying of an esomeprazole sodium formulation has been conducted in a conventional lab-scale spray-dryer from a water solution of the formulation. The spray drying is conducted with a rather high inlet air temperature. Even, if the substance is sensitive to heat, a high temperature of the inlet air could be used. A possible explanation would be that the substance/formulation would withstand this inlet temperature due to the fact that water will evaporate from the substance/formulation during this drying step and cool down the substance/formulation and the exposure time in the inlet air stream is very short.

According to one aspect of the preparation process, the dissolved components are passed through a particle retention filter before the solution is spray-dried. The filtering step may be advantageous to avoid particles in the formulation. The spray-drying step may provide additional advantages to the solid formulation, such as enhanced powder properties, e.g. controlled particles size and density and enhanced dissolution properties of the powder.

The spray drying may be performed aseptically to provide a solid formulation essentially free from particles, such as any particular matter from the preparation of the proton pump inhibitor compound. Hence, the spray-dried material is suitable for an ex tempore preparation of a solution for parenteral administration. According to another embodiment of the present invention the non-lyophilized solid formulation is spray-dried before it is filled in its final container and sterilized by radiation.

Suitable final containers for the present invention are multi-compartment systems, such as two-chamber infusion bags and two-compartment syringes. These containers may also be provided with a particle filter, i.e. that the solution for parenteral administration is filtered in the device before administered to the body.

For example, if the container is a two-chamber container such as an infusion bag, one of the chambers is filled with the solid formulation and the other chamber is filled with a suitable solvent and a weak seal separates the two chambers. The solvent may optionally comprise pharmaceutically acceptable inactive excipients, such as excipients that control the pH of the final solution.

The whole container, i.e. the parenteral formulation in its final container, is then sterilized by ionizing radiation. The sterilized infusion bag is an “easy to use” ex tempore preparation product for parenteral administration.

Alternatively, the stable solid formulation is first prepared and then sterilized by ionization radiation before aseptic filling of the formulation into a container, optionally together with a sterile solvent, which solvent has been pre-filled into a separate compartment.

Thus, the present invention provides a sterilized parenteral formulation in its final container for ex tempore preparation of a solution for parenteral administration without using lyophilisation processes/steps in the manufacturing.

The manufactured parenteral formulation in its final container with the sterilized solid composition in one compartment and optionally with a reconstitution solvent in a second compartment can be stored in room temperature (See Example 1, Table 1) or at elevated temperatures (e.g. 40° C./75% RH) for at least 12 months without significant degradation of the active ingredient (See Example 1, Table 2). The sterilized solid formulation may also be stored under the same conditions without significant degradation.

The present invention also relates to the use of any of product according to the present invention, such as a sterilized parenteral formulation in its final container or a sterilized solid formulation, in medicine. The pharmaceutical active compounds used in the claimed sterilized parenteral formulations or sterilized solid formulation are useful for inhibiting gastric acid secretion in mammals including man by controlling gastric acid secretion at the final step of the acid secretory pathway and thus reduce basal and stimulated gastric acid secretion irrespective of stimulus.

The pharmaceutical active compounds used in the present invention are effective as gastric acid secretion inhibitors, and are thus useful as antiulcer agents. In a more general sense, they can be used for prevention and treatment of gastric-acid related conditions in mammals and especially in man, including e.g. reflux esophagitis, gastritis, duodenitis, gastric ulcer and duodenal ulcer. Furthermore, they may be used for treatment of other gastrointestinal disorders where gastric acid inhibitory effect is desirable e.g. in patients on NSAID therapy, in patients with Non Ulcer Dyspepsia, in patients with symptomatic gastro-esophageal reflux disease, and in patients with gastrinomas. They may also be used in patients in intensive care situations, in patients with acute upper gastrointestinal bleeding, pre- and postoperatively to prevent aspiration of gastric acid, to prevent and treat stress ulceration and asthma, and for improvement of sleep. Further, the compounds of the invention may be useful in the treatment of psoriasis as well as in the treatment of Helicobacter infections and related diseases. The compounds of the invention may also be used for treatment of inflammatory conditions in mammals, including man.

In the practice of the invention, the magnitude of the therapeutic dose will depend on the nature and severity of the disease to be treated. The dose, and dose frequency, may also vary according to the age, body weight and response of the individual patient. Special requirements may be needed for patients having Zollinger-Ellison syndrome, or Peptic Ulcer Bleed such as a need for higher doses than the average patient. Children and patients with liver diseases generally will benefit from doses that are somewhat lower than the average. Thus, in some conditions it may be necessary to use doses outside the ranges stated below, for example long-term treatments may request lower dosage. Such higher and lower doses are within the scope of the present invention. Daily doses may vary between 5 mg to 300 mg. Suitable doses for injection and infusion comprise for instance 5, 10, 15, 20, 30, 40, 60, 80 and 100 mg of the pharmaceutical active compound.

Combination preparations and combination therapies comprising the pharmaceutical active proton pump inhibitor compounds and other active ingredients may also be used. Examples of such other active ingredients include, but are not limited to anti-bacterial compounds, non-steroidal anti-inflammatory agents (NSAID) such as acetyl salicylic acid, diclofenac, naproxen and COX-2 agents, antacid agents, alginates, prokinetic agents, motility stimulating drug, and a H₂ blocker, such as for instance ranitidine.

For the avoidance of doubt, “treatment” includes the therapeutic treatment, as well as the prophylaxis, of a condition.

The present invention also relates to the use of the formulation as disclosed above in the manufacture of a medicament to be used in the treatment of gastrointestinal diseases.

The present invention also relates to a method for preventing and treating gastrointestinal diseases wherein any one of the stable solid formulations according to the invention is administered to a subject in the need thereof.

### EXAMPLES

In the following the invention has been described by non-limiting examples of formulations comprising four acid susceptible proton pump inhibitors, omeprazole, pantoprazole, lansoprazole and esomeprazole with and without a pharmaceutically acceptable excipient, such as the inactive ingredient lactose, which formulations have been sterilized by gamma or electronic beam radiation. Also included are examples on e-beam radiated spray-dried solid formulations comprising sodium salt of esomeprazole with and without a pharmaceutically acceptable excipient such as the inactive ingredient sodium chloride. The formulations were compared with a lyophilized formulation (non gamma sterilized) and the non-gamma sterilized esomeprazole sodium substance (dry powder). The results show a good stability of the claimed gamma or electronic beam sterilized solid formulations of the invention.

Example 5 exemplifies a suitable route for preparation of esomeprazole sodium.

### Example 1

### Stable Gamma Sterilized Formulations of Esomeprazole Sodium

Three different gamma sterilized formulations of esomeprazole sodium (A-C) were analyzed after different storage times at room temperature. Formulations A-B comprised esomeprazole sodium (dry powder) filled in glass vials. Formulation C comprised a mixture of esomeprazole sodium and lactose 15:85% w/w (dry powder). The sterilizing dose used was 25 kGy. Non-gamma sterilized esomeprazole sodium drug substance (D) was used as reference. The appearance of the powder was determined after different storage times.

TABLE 1
Appearance and organic impurities of different esomeprazole
formulations, stored at 25° C.

| | A | B | C | D |
|---|---|---|---|---|
| Formulation | Esomeprazole | Esomeprazole | Esomeprazole | Esomeprazole |
| | sodium | sodium | sodium:Lactose | sodium |
| | | | (15:85% w/w) | |
| Package | Tube with screw | Vial, | Tube with screw | Double LDPE- |
| | cap, | glass type I¹ | cap, | bags inside a |
| | glass type I¹ | (radiation | glass type I¹ | welded |
| | | resistant) | | aluminum bag |
| Gamma radiated | Yes | Yes | Yes | No |
| (sterilizing dose of | | | | |
| 25 kGy) | | | | |

Storage time - 0 months

| | | | | |
|---|---|---|---|---|
| Appearance | Very slightly | Very slightly | Slightly yellow | White to almost |
| | yellow | yellow | | white |
| Organic impurities, | <0.1 | <0.1 | 0.2 | <0.1 |
| total (area %) | | | | |

Storage time - 12 months

| | | | | |
|---|---|---|---|---|
| Appearance | Slightly yellow | Slightly yellow | Yellow | White to almost |
| | | | | white |
| Organic impurities, | <0.1 | <0.1 | <0.1 | <0.1 |
| total (area %) | | | | |

¹Glass type I is neutral glass with a high hydrolytic resistance due to the chemical formulation of the glass itself, as defined in the European Pharmacopoeia (Ph Eur 2005)

As shown in Table 1, the gamma-sterilized formulations A-C remain stable after radiation and the amount of organic impurities are in the same range as the non-radiated esomeprazole sodium (D). Some small color changes of the formulations after radiation could be observed.

The importance of using gamma radiation resistant and properly sealed containers for the described formulations (A-C) is shown in Table 2. When non-radiation resistant glass type I is radiated, the glass turns brown after radiation. Radiation resistant glass remains uncolored. The formulation radiated in the non-radiation resistant glass tube became black and showed a high amount of organic impurities when stored in the accelerated climate 40° C./75% RH. This effect results most likely from improper (not tight) sealing of the tube rather than an effect of the glass material itself.

When gamma radiation resistant glass, with a proper sealing, is used, the formulation remains stable even after 12 months in 40° C./75% RH, which must be considered to be unexpected due to the known liability of acid susceptible proton pump inhibitors against heat and moisture.

TABLE 2
Appearance and organic impurities of a radiated esomeprazole
formulation packed in two different glass vials, stored at 40° C./75% RH

| | A | B |
|---|---|---|
| Formulation | Esomeprazole sodium | Esomeprazole sodium |
| Package | Tube with screw cap, | Vial, |
| | glass type I¹ | glass type I¹ |
| | | (radiation resistant) |
| Gamma radiated | Yes | Yes |
| (sterilizing dose of 25 | | |
| kGy) | | |
| Appearance of package | Brown | Uncolored |
| after radiation | | |

Storage time - 0 months

| | | |
|---|---|---|
| Appearance | Very slightly yellow | Very slightly yellow |
| Organic impurities, | <0.1 | <0.1 |
| total (area %) | | |

Storage time - 12 months

| | | |
|---|---|---|
| Appearance | Black | Yellow |
| Organic impurities, | 3.3 | 0.2 |
| total (area %) | | |

¹Type I glass is neutral glass with a high hydrolytic resistance due to the chemical formulation of the glass itself, as defined in the European Pharmacopoeia (Ph Eur 2005)

### Example 2

### A Gamma Sterilized Lyophilized Formulation of Esomeprazole Sodium

A lyophilized formulation (E) was sterilized with gamma radiation (25 kGy). The appearance and the total amount of organic impurities after radiation was compared with a non-gamma radiated formulation (F).

TABLE 3
Appearance and organic impurities of a lyophilized
esomeprazole (20 mg) formulation after gamma radiation with 25 kGy

| | E | F |
|---|---|---|
| Formulation | Esome Na (incl EDTA) | Esome Na (incl EDTA) |
| Package | Vial, | Vial, |
| | glass type I¹ | glass type I¹ |
| Gamma radiated | Yes | No |
| (sterilizing dose of | | |
| 25 kGy) | | |
| Appearance | Slightly green | White to off-white |
| Organic impurities, | 0.4 | 0.2 |
| total (area %) | | |

¹Type I glass is neutral glass with a high hydrolytic resistance due to the chemical formulation of the glass itself, as defined in the European Pharmacopoeia (Ph Eur 2005)

As shown in Table 3 some small color changes and minor degradation could be observed.

### Example 3

### Gamma Sterilized Formulations of Three Acid Susceptible Proton Pump Inhibitors

In addition to esomeprazole sodium exemplified in Example 1, three other acid susceptible proton pump inhibitors, omeprazole sodium, pantoprazole sodium and lansoprazole, were gamma sterilized with a sterilizing dose of 25 kGy. The appearance of the powder was determined before and after gamma sterilization.

TABLE 4
Appearance of three acid susceptible proton pump inhibitors
before and after gamma sterilization

| Proton pump inhibitor | Appearance | |
|---|---|---|
| (powder) | Before sterilization | After sterilization (25 kGy) |
| Omeprazole sodium | White to off-white | Very slightly yellow |
| Pantoprazole sodium | White to off-white | Very slightly yellow |
| Lansoprazole | Very slightly | Very slightly |
| | yellowish-brown | yellowish-brown |

As in example 1 and 2 some small (very minor) color changes could be observed after radiation for two of these formulations, i.e. omeprazole sodium and pantoprazole sodium, but no color change was observed for lansoprazole.

### Example 4

### Stable Electronic Beam Radiated Formulations of Esomeprazole Sodium

Three different formulations of esomeprazole sodium (G-I) were sterilized with electronic beam radiation corresponding to a dose of about 25 kGy. Formulation G comprised esomeprazole sodium drug substance (dry powder), formulation H comprised spray-dried esomeprazole sodium (dry powder) and formulation I comprised a spray-dried 50:50% w/w mixture of esomeprazole sodium and sodium chloride (dry powder). The spray-dried formulations were obtained by first dissolving the dry esomeprazole sodium powder (either with or without excipient) in water and then drying the formulation in a lab-scale spray-dryer using co-current flow and a two-fluid nozzle. The inlet temperature was about 170° C. and the outlet temperature about 80-90° C.

All formulations were packed in small polypropylene plastic bags, which were placed inside aluminum bags. The appearance of the powder and the total amount of organic impurities was determined before and after radiation.

TABLE 5
Appearance and organic impurities of different esomeprazole
formulations before and after radiation

| | G | H | I |
|---|---|---|---|
| Formulation | Esomeprazole sodium | Esomeprazole sodium, | Esomeprazole |
| | | spray-dried powder | sodium:sodium chloride |
| | | | (50:50% w/w), |
| | | | spray-dried powder |

Before radiation

| | | | |
|---|---|---|---|
| Appearance | White to off-white | Off-white | Off-white |
| Organic impurities, | <0.1 | <0.1 | <0.1 |
| total (area %) | | | |

After electronic beam radiation (25 kGy)

| | | | |
|---|---|---|---|
| Appearance | Off-white, slightly | Off-white, slightly | Off-white, slightly |
| | colored | colored | colored |
| Organic impurities, | <0.1 | 0.1 | 0.1 |
| total (area %) | | | |

The results in Table 5 are very similar to what was obtained after gamma sterilization hence both types of radiation can be used. As in example 1-3 some small color change could be observed.

### Example 5

### Preparation of Esomeprazole Sodium

Esomeprazole sodium may be prepared by using the process described in WO 96/02535 hereby incorporated by reference.

It may also be prepared by using esomeprazole potassium as starting material. Esomeprazole potassium may be prepared as described in WO 98/54171 hereby incorporated by reference.

Preparation of Esomeprazole Sodium from Esomeprazole Potassium.

Acetic acid and water is added to a stirred suspension of esomeprazole potassium in toluene, whereby esomeprazole dissolve in the organic phase. The organic phase is washed with brine. Esomeprazole sodium is precipitated by addition of methanol followed by aqueous sodium hydroxide. The crude product is isolated and washed with toluene. Finally, the crude product of esomeprazole sodium is recrystallized in water/acetone using acetonitril as anti-solvent. The pure product is isolated, washed with acetonitril and dried.

1. A stable sterilized parenteral formulation comprising a solid formulation comprising an acid susceptible proton pump inhibitor and optionally pharmaceutically acceptable excipients wherein said formulation has been sterilized in its final container by ionizing radiation.

2. A formulation according to claim 2, wherein said acid susceptible proton pump inhibitor is water-soluble.

3. A formulation according to any one of claims 1 or 2, wherein said ionizing radiation is selected from the group of gamma and electronic beam radiation.

4. A formulation according to any of claims 1-3, wherein the container is a multi-chamber container and one compartment comprises the stable solid formulation and a second compartment comprises a solvent.

5. A formulation according to any one of claims 1-4, wherein said container has a particle filter incorporated in its construction.

6. A formulation according to any one of claims 1-5, wherein said acid susceptible proton pump inhibitor is selected from Formula I 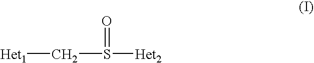
wherein
Het₁ is 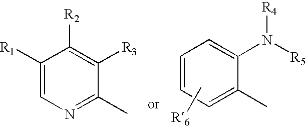
Het₂ is 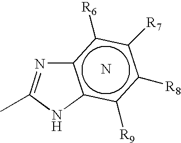
wherein
N in the benzimidazole moiety means that one of the carbon atoms substituted by R₆-R₉ optionally may be exchanged for a nitrogen atom without any substituents;
R₁, R₂ and R₃ are the same or different and selected from hydrogen, alkyl, alkoxy optionally substituted by fluorine, alkylthio, alkoxyalkoxy, dialkylamino, piperidino, morpholino, halogen, phenyl and phenylalkoxy;
R₄ and R₅ are the same or different and selected from hydrogen, alkyl and aralkyl;
R′₆ is hydrogen, halogen, trifluoromethyl, alkyl and alkoxy;
R₆-R₉ are the same or different and selected from hydrogen, alkyl, alkoxy, halogen, halo-alkoxy, alkylcarbonyl, alkoxycarbonyl, oxazolyl, pyrrolyl, trifluoroalkyl, or adjacent groups R6-R9 form ring structures;
or one of its single the enantiomer thereof,
and in the above definitions the alkyl groups, alkoxy groups and moieties thereof, may be branched or straight C₁-C₉-chains or comprise cyclic alkyl groups, such as cycloalkylalkyl.

7. A formulation according to any one of claims 1-6, wherein said compound of formula I is in the form of a pharmaceutically acceptable salt or in its neutral form.

8. A formulation according to claim 6, wherein said compound of the general formula I is selected from a sodium or a potassium salt of either

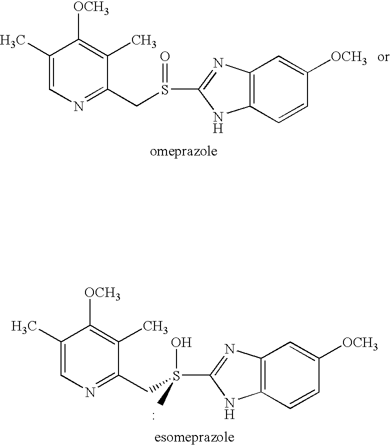

9. A formulation according to any of one of claims 1-7, wherein said container is a container resistant against gamma or electronic beam radiation.

10. A stable solid formulation comprising an acid susceptible proton pump inhibitor and optionally pharmaceutically acceptable excipients wherein said formulation has been sterilized by ionizing radiation.

11. A formulation according to claim 10, wherein said acid susceptible proton pump inhibitor is water-soluble.

12. The formulation according to any one of the preceding claims 10 or 11, wherein said ionizing radiation is selected among gamma radiation and electronic beam radiation.

13. The formulation according to any one of claims 10-12, wherein said acid susceptible proton pump inhibitor is selected from a compound with the general formula I as defined in claim 6.

14. The formulation according to any one of claims 10-13, wherein said compound of formula I is in the form of a pharmaceutically acceptable salt or in its neutral form.

15. The formulation according to claim 13, wherein said compound of the formula I is selected from a sodium or a potassium salt of

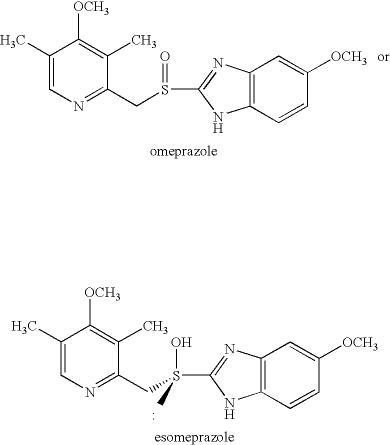

16. A solution for parenteral administration comprising the stable solid formulation according to any one of claims 10-15 together with a solvent.

17. A process for the preparation of a formulation according to any one of claims 10-15, wherein the acid susceptible proton pumps inhibitor is optionally mixed with pharmaceutically acceptable excipients and thereafter optionally dissolved in a suitable solvent, e.g. water or ethanol and dried by spray-drying, and finally the formulation is sterilized with ionization radiation.

18. A process according to claim 17, wherein said ionizing radiation is selected from the group of gamma and electronic beam radiation.

19. A process for the manufacture of a product comprising a formulation according to any one of claims 1-9, comprising the following steps:
(i) filling a container with a formulation comprising an acid susceptible proton pump inhibitor and optionally pharmaceutically acceptable excipients; and
(ii) sterilizing the filled container by using ionizing radiation.

20. A process according to claim 19, wherein said ionizing radiation is selected from the group of gamma and electronic beam radiation.

21. The process according to any one of claims 19-20, wherein the ionizing radiation has an absorbed minimum dosage of up to about 45 kGy.

22. The process according to claim 21, wherein the ionizing radiation has an absorbed dosage in the range 10 to 40 kGy.

23. The process according to claim 21, wherein the ionizing radiation has an absorbed dosage of about 25 kGy.

24. The formulation according to any one of the claims 1-9 or the stable solid composition according to any one of claims 10-15 for use in medicine.

25. A method for preventing or treating gastrointestinal diseases wherein the stable solid composition according to any of claims 10-15 after reconstitution with an aqueous solvent is administered to a subject in the need of such treatment.

26. (canceled)
